(19) United States
(12) Patent Application Publication
(10) Pub. No.: US 2013/0060134 A1
(43) Pub. Date: Mar. 7, 2013

(54) CZT SENSOR FOR TUMOR DETECTION AND TREATMENT

(71) Applicants: Dennis ESHIMA, Phoenix, AZ (US); Mehmet HUSNU, Phoenix, AZ (US); Jim STONE, Alpharetta, GA (US)

(72) Inventors: Dennis ESHIMA, Phoenix, AZ (US); Mehmet HUSNU, Phoenix, AZ (US); Jim STONE, Alpharetta, GA (US)

(73) Assignee: CARDINAL HEALTH 414, LLC, Dublin, OH (US)

(21) Appl. No.: 13/606,591

(22) Filed: Sep. 7, 2012

Related U.S. Application Data
(60) Provisional application No. 61/531,805, filed on Sep. 7, 2011.

(51) Int. Cl. G01T 1/24; A61B 6/04; A61N 5/10; G01T 1/167

(52) U.S. Cl. 600/431; 250/336.1; 250/394; 250/370.13; 600/1

(57) ABSTRACT

A tumor treatment apparatus may include an array of collimated CZT detectors configured to intersect a known coordinate and measure gamma radiation activity, for example at 511 keV. A radiation delivery system may be configured to direct radiation through the known coordinate on the basis of the gamma radiation activity. A translatable and rotatable table may be configured to support a tumor host, wherein the tumor is positionable relative to the known coordinate on the basis of the gamma radiation activity emitted by the tumor and measured by the array of collimated CZT detectors. Radiation from the radiation delivery system may be delivered to the tumor at the known coordinate, and may be delivered in an intra-operative surgical environment.

This application claims priority from U.S. Patent Application No. 61/531,805, titled “CZT Sensor for Tumor Detection and Treatment,” filed on Sep. 7, 2011, and which is incorporated herein by reference in its entirety.

### FIELD OF INVENTION

Aspects of the current invention relate to gamma ray sensors. Particularly, aspects of the current invention relate to a method and apparatus for detecting tumors tagged with radiopharmaceuticals, for example PET radiopharmaceuticals emitting radiation at 511 keV, for gamma ray and/or proton therapy radioisotope using cadmium zinc telluride (CZT) solid state detectors.

### BACKGROUND

Diagnostic techniques in nuclear medicine typically use radioactive tracers that emit gamma rays from within the body of a patient. These tracers are generally short-lived isotopes linked to chemical compounds that permit specific physiological processes to be studied, and can be given to the patient via injection, via inhalation or orally. In one type of diagnostic technique, for example single photons are detected by a gamma ray sensitive camera, which can view organs from many different angles. The camera builds up an image from the points from which radiation is emitted, and the image is enhanced by a computer and viewed by a physician on a monitor for indications of abnormal conditions.

A more recent development is Positron Emission Tomography (PET), which is a more precise technique using isotopes produced in a cyclotron, in which protons are introduced into the nucleus of the isotope, which results in a deficiency of neutrons (i.e., the isotope becomes proton rich). Positron emission tomography (PET) is a nuclear medicine imaging technique that produces a three-dimensional image or picture of functional processes within the body of the patient. The system detects pairs of gamma rays emitted indirectly by a positron-emitting radionuclide (tracer), which is introduced into the body on a biologically active molecule. Three-dimensional images of tracer concentration within the body are then constructed by computer analysis. In modern scanners, three dimensional imaging is often accomplished with the aid of a computed tomography (CT) X-ray scan performed on the patient during the same session. As can be seen in FIG. 1, a typical PET device of the related art dedicated to diagnostic imaging is fairly large. When imaging a tumor that may be treated surgically or by directed gamma or proton therapy, a patient's body is typically marked externally (via bone features, or other externally selected sites), often with laser targeting, so that the patient may be properly placed for accurate radiation treatment aimed at the tumor location, while attempting to minimize radiation damage to adjacent normal tissue. However, there are drawbacks with such a configuration. For example, because it may not be feasible to conduct surgery in an operating room simultaneously with a system as large and intrusive to the surgical theater at the PET equipment illustrated in FIG. 1, the patient is often relocated to an operating theater at a later time. Therefore, there is the possibility that the tumor location determined by coordinating PET imaging with external body markers is only approximately accurate when the patient is moved during the period between imaging and treatment, and the tumor may actually shift relative to the imaged location, which results in poor targeting of the tumor and a decreased efficiency of the cancer treatment.

For use in such imaging devices, the nucleus of a radioisotope usually becomes stable by emitting an alpha and/or beta particle (or a positron), which may be accompanied by the emission of energy in the form of electromagnetic radiation known as gamma rays. This process is known as radioactive decay.

A positron-emitting radionuclide is introduced in the body of a patient, usually via injection, and accumulates in the target tissue of the body of the patient. As the radionuclide decays, the radionuclide emits a positron, which promptly combines with a nearby electron in the target tissue of the body of the patient, resulting in the simultaneous emission of two identifiable gamma rays in opposite directions, each having an energy of 511 keV. These are conventionally detected by a PET camera and give very precise indication of their origin. PET's most important clinical role is in oncology, with Fluorine-18 (F-18) as the tracer, since it has proven to be the most accurate non-invasive method of detecting and evaluating most cancers. Fluorine-18 is one of several cyclotron producible positron emitters, along with Carbon-11, Nitrogen-13, and Oxygen-15, that are used in PET for studying brain physiology and pathology, in particular for localizing epileptic focus, and in dementia, psychiatry and neuropharmacology studies. These positron emitters also have a significant role in cardiology.

When the biologically active molecule chosen for PET is FDG (fluorodeoxyglucose), an analogue of glucose, the concentrations of tracer images generally give a spatially observable indication of tissue metabolic activity in the form of regional glucose uptake. Tumors may have higher metabolic activity than normal surrounding tissue, and therefore exhibit greater uptake of FDG than they would with normal glucose. With respect to cancer detection and therapy, F-18 in FDG has become very important in detection of cancers having elevated glucose metabolisms and the monitoring of progress in their treatment, using PET. A radioactive product such as F-18 in FDG is known as a radiopharmaceutical. Other types of cancer may show an elevated metabolism of different molecules, which therefore may be synthesized with an appropriate radionuclide.

F-18 has a half-life of approximately 110 minutes, which is beneficial in that it does not pose a long-term environmental and health hazard. For example, after 24 hours, the radioactivity level is approximately 0.01% of the product when freshly produced in a cyclotron. Consequently, there is typically no significant long-term hazard either to the patient or the environment, because the decay rate is rapid and short-term.

Whereas PET cameras are effective in imaging uptake of F-18 in FDG, they are typically too large and ineffective for in situ surgical or radiation treatment settings. There is a need in the art, therefore, for a method and apparatus to timely detect tumor location for accurate treatment without having to resort to reliance on imaging data that is only approximately accurate given the time interruption between imaging and treatment. It may further be beneficial to provide a sensing apparatus for tumor detection and imaging that may be used in the same theater as a treatment apparatus. It may also be beneficial if the sensing apparatus can be used to monitor tumor activity during the treatment process.

### SUMMARY

in light of the above described problems and unmet needs, aspects of methods and systems for detecting radioisotope concentration, activity and sample volume, are provided.

According to various aspects of the current invention, a gamma ray detector may include a gamma ray detecting rod elongated in one direction to a specified length, and a gamma ray shield encapsulating the rod, the shield having an opening opposite an end of the elongated rod to admit gamma rays substantially parallel to the longitudinal axis of the elongated rod, wherein the longitudinal axis of the rod and the opening are directable toward a volume of gamma ray emitting material observable by the detector on the basis of the length of the elongated rod and the opening in the gamma ray shield.

According to other aspects of the current invention, an apparatus for detecting gamma ray emissions from a tumor may include one or more sensors to image the tumor for guiding a radiation treatment device to selectively deliver a measured dose to the tumor on the basis of the image provided by the sensor.

To the accomplishment of the foregoing and related ends, one or more aspects of the current invention include the features hereinafter fully described and particularly pointed out in the claims. The following description and the annexed drawings set forth in detail certain example features of the one or more aspects. These examples provide, however, but a few of the various ways in which the principles of various aspects may be employed and the described aspects are intended to include all such aspects and their equivalents.

Additional advantages and novel features of these aspects of the invention will be set forth in part in the description that follows, and in part will become more apparent to those skilled in the art upon examination of the following or upon learning by practice of aspects of the invention.

### BRIEF DESCRIPTION OF THE DRAWINGS

These and other sample aspects of the invention will be described in the detailed description that follow, and in the accompanying drawings, wherein:

FIG. 1 is an image of an example related art positron emission tomography (PET) device;

FIG. 2A is a representative diagram of various features of a CZT gamma ray detector in accordance with aspects of the current invention;

FIG. 2B is a plan view of the conceptual illustration of the CZT gamma ray detector of FIG. 2A;

FIG. 3 is a representative illustration of an example circuit for measuring gamma rays with the detector of FIGS. 2A and 2B, according to aspects of the current invention;

FIG. 4 is a representative diagram for an example apparatus for detecting and imaging tumors absorbing a radiopharmaceutical using the detector and circuitry of FIGS. 2-3 in coordination with a radiation delivery therapy system, according to aspects of the current invention;

FIG. 5 is a flowchart of an example process of imaging a patient tumor according to aspects of the current invention;

FIG. 6 is a flowchart of an example process of beam therapy delivery guided by imaging detection of an array of one or more CZT collimated detectors, according to aspects of the current invention;

FIG. 7 is a representative diagram for an example apparatus for detecting and imaging tumors with an absorbed radiopharmaceutical using the detector and circuitry of FIGS. 2-3 in coordination with a surgical excision procedure, according to aspects of the current invention;

FIG. 8 is an example system diagram of various hardware components a the computing system and other features for use in networking the apparatus of FIG. 4; and

FIG. 9 is a block diagram of various example system components for providing communications over a network with and between various components of the apparatus of FIG. 4 for detecting and treating tumors, in accordance with an aspect of the present disclosure.

In accordance with common practice, some of the drawings may be simplified for clarity. Thus, the drawings may not depict all of the components of a given apparatus (e.g., device) or method. Like reference numerals may be used to denote like features throughout the specification and figures.

### DETAILED DESCRIPTION

Various aspects of methods and apparatuses are described more fully hereinafter with reference to the accompanying drawings. These methods and apparatus may, however, be incorporated in many different forms and should not be construed as limited to any specific structure or function presented throughout this disclosure. Rather, these aspects are provided so that this disclosure will be thorough and complete, and will fully convey the scope of these methods and apparatuses to those skilled In the art. Based on the description herein, one skilled in the art will appreciate that that the scope of the disclosure is intended to cover any aspect of the methods and apparatuses disclosed herein, whether implemented independently of or combined with any other aspect(s) of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure herein may be incorporated within one or more elements of a claim.

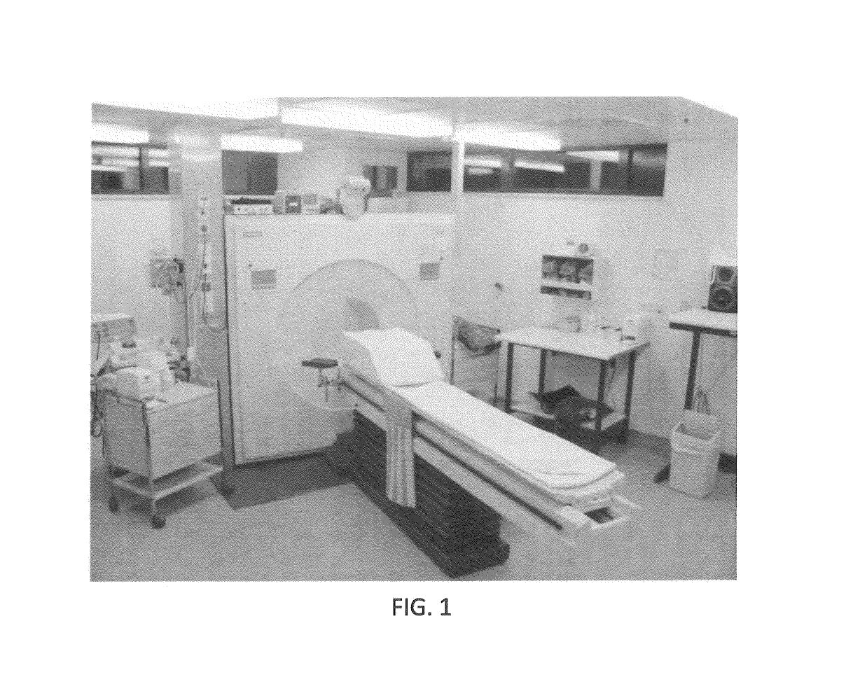

FIG. 1 is an image of an example positron emission tomography (PET) device of the related art. In a positron emission tomography (PET) device as illustrated in FIG. 1, for example, a patient is injected with a radiopharmaceutical tagged with a radionuclide, which may typically be FDG—a glucose-like molecule. After a short period, the FDG metabolizes and concentrates more in tissues with higher metabolic activity, such as cancer tumors, than in normal tissue. A body marker, such as a laser or other pointing system may then be used to mark locations on the patient's body that correspond to the higher concentrations of FDG, and thus to cancer tumors, so that, at a later time, a gamma ray, proton or carbon beam radiation treatment facility (or other equivalent radiation treatment system) can aim the radiation beam at the tumors detected via the PET system using the body marker as a guide. However, because the patient is often moved from the imaging facility to the treatment facility, the accuracy of locating the tumor by coordinating with the body marker is not ideally reliable.

In the PET device shown, the radioactive isotope F-18, which is contained in FDG, emits a positron that reacts with an electron in the tissue of the patient to produce two gamma rays in approximately opposite directions, each having an energy of 511 keV. The apparatus according to various aspects of the current invention, may be used to locate and determine the extent and size of body portions, such as tumors, using one or more gamma ray detectors in a configuration that is less intrusive than a full PET system of the related art, and may therefore also be used contemporaneously, for example, with a therapeutic radiation beam treatment system to guide the beam more accurately than in conventional systems. This greater accuracy may result, for example, from the detection/location/imaging using one or more gamma ray detectors contemporaneously with treatment.

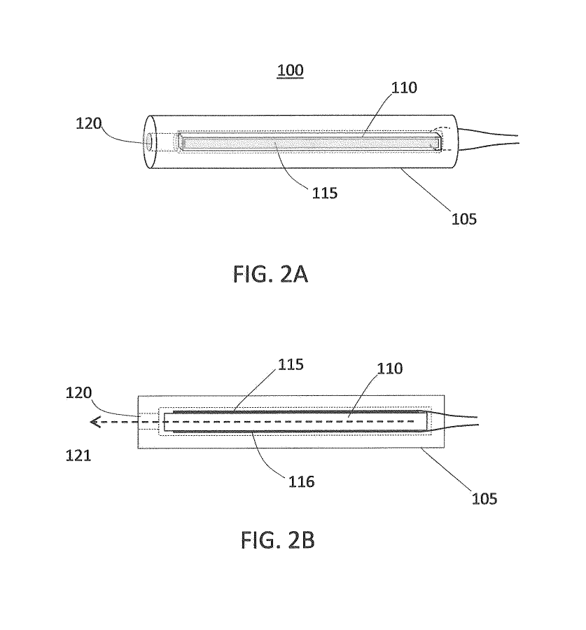

FIG. 2A is a representative drawing of an example CZT gamma ray detector in accordance with aspects of the current invention, and FIG. 28 is a representative schematic side-view of the CZT gamma ray detector of FIG. 2A, as viewed along a longitudinal axis 121 of the detector 100. According to various aspects, the detector 100 may include a cadmium zinc telluride (CdZnTe, or CZT) element 110; however, other solid state materials with similar characteristics currently available or yet to be discovered may be similarly used. CZT is a direct bandgap semiconductor and can operate in a direct-conversion (e.g., photoconductive) mode at room temperature, unlike some other materials (e.g., germanium) which may require cooling, in some cases, to liquid nitrogen temperatures. One relative advantages of CZT over other detectors, such as germanium, include a high sensitivity for x-rays and gamma-rays, due to the high atomic numbers and masses of Cd and Te relative to other detector materials typically used in the related art, and better energy resolution than scintillator detectors. In operation, a gamma ray (photon) traversing a CZT element 110 liberates electron-hole pairs in its path. A bias voltage applied across electrodes 115 (not shown in FIGS. 1) and 116 on the surface of the element 110 (both shown in a side view in FIG. 2) causes the charges to be swept to the electrodes 115 and 116 on the surface of the CZT, where the electrons move toward the anode and the holes (protons) move toward the cathode. Wires or other suitable couplings may connect the electrodes 115 and 116 to a source of the applied voltage.

According to various aspects, the detector 100 can function accurately as a spectroscopic gamma energy sensor, particularly when the element 110 is CZT. However, geometric aspects may be considered. In related art use of CZT as a gamma ray detector, the CZT element 110 may be a thin platelet, sometimes arranged in multiples to form arrays for imaging, facing the source of gamma ray emission. Therefore, gamma rays of differing energies may traverse a detector element of substantially the same thickness. While absorption of the gamma ray may generally be less than 100% efficient, higher energy gamma rays will liberate more electron-hole pairs than lower energy gamma rays, producing a pulse of greater height. The spectrum and intensity of gamma ray energies may thus be spectroscopically determined by counting the number of pulses generated corresponding to different pulse heights.

According to various aspects, because higher energy photons may travel greater distance in the CZT rod 110 before complete absorption, it is advantageous for the CZT rod 110 to be greater in length in a direction (i.e., the longitudinal axis direction 121), in order to intersect a volume of a tumor containing the radiopharmaceutical being detected. Gamma rays incident on the CZT rod off axis or transverse to the longitudinal axis direction 121 may not be fully absorbed, and may not be as sensitive at detection as a result. Thus, elongating the CZT rod 110 in the one longitudinal axial direction 121 introduces a degree of collimation and directional sensitivity along the axis direction 121.

The absorption coefficient for 511 keV gamma ray absorption in CZT is μ=0.0153 cm²/gm. The absorption probability as a function of μ, density ρ (=6.34 gm/cm³) and penetration distance h is

P(μ, h)=1−e^{-μρh}.

Therefore, the ratio of absorption in a 10 mm length of CZT to a 1 mm length is

$$\frac{P ( μ , 10 mm )}{P ( μ , 1 mm )} \sim 9.613 .$$

That is, the directional sensitivity for gamma ray detection of CZT at 511 keV along the 10 mm length of the longitudinal axis direction 121 of the detector is nearly 10 times greater than in the 1 mm thick transverse direction. According to various aspects, the ratio of the length of the CZT detector 110 to the diameter of the CZT detector 110 may be anywhere between ⅕ and 1/200 with increments corresponding to the diameter of the CZT detector 110, i.e., ⅕, ⅙, 1/7 . . . 1/200.

Referring to FIGS. 2A-2B, according to various aspects, the sensor may comprise a CZT rod 110 as described above, encased in a shielded case 105 (e.g., a tungsten case) with an aperture 120 open and directed toward the radioactive material to expose the CZT rod 110 only along the longitudinal axis direction 121 of the rod 110. According to various aspects, the shielded case 105 allows exposure of the longitudinal axis direction 121 of the rod 110, while shielding or inhibiting the CZT rod 110 from gamma rays incident laterally or transverse to the longitudinal axis direction 121 of the rod 110, i.e., from directions other than along the long longitudinal axis direction 121. The relative dimensions of the CZT rod 110, shield 105 and aperture 120 illustrated in FIGS. 2A-2B are not necessarily shown to scale, and may be varied, as discussed above. Therefore, the combination of the shielding case 105, the aperture 120 and the extended length of the CZT detector in the direction of gamma ray emission from a portion of the radiation source, such as a radiopharmaceutical rich tumor provides a substantial directional “virtual” collimation of the CZT rod 110 sensitivity to gamma rays radiating from the tumor. In addition, the “virtual collimation” provides the ability to discriminate the energy by using high speed signal processing and filtering out secondary lower energy radiation from the surrounding tissue. As a result, by properly scanning the tumor region to detect emitted gamma rays, either by movement of one or more detectors 100 and/or the patient, or both, the tumor may be imaged and spatially identified.

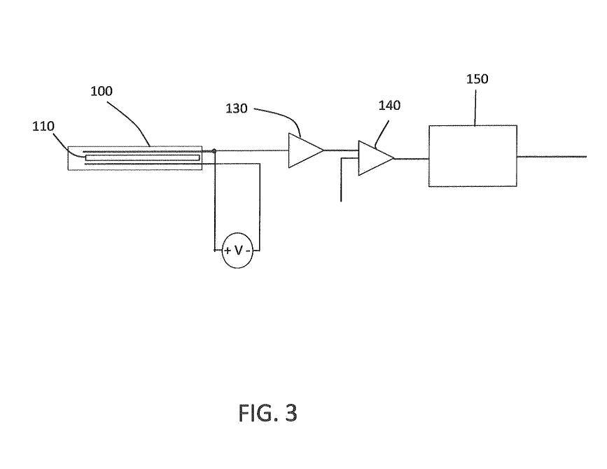

FIG. 3 is a representative illustration of an example circuit for measuring gamma rays with the detector of FIGS. 2A and 2B, according to aspects of the current invention. A charge amplifier 130 may be coupled to the electrodes 115 and 116 to amplify a charge. A pulse generator 140 may convert the sensed charge to a pulse, where the pulse height may be proportional to the energy of the gamma ray. A counting circuit 150 such as, for example, a high speed field programmable learning array (FPLA) that discriminates signal such as 511 keV gamma emissions as a function of energy and activity, may determine the number of pulses as a function of energy, therefore, the detector 100 may function as a spectroscopic tool that is capable of measuring the radioactivity as a function of gamma ray energy.

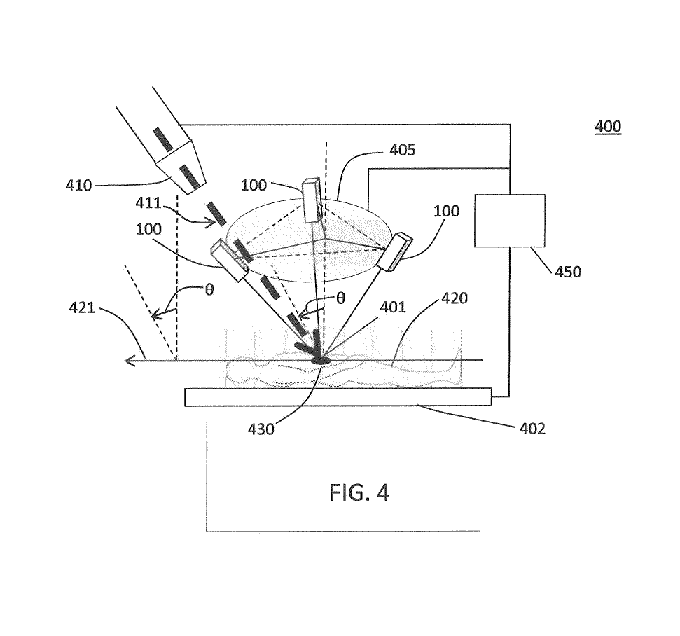

FIG. 4 is a representative illustration of an example apparatus 400 for detecting and imaging a radiation source, such as a radiation emitting part of a patient's body (e.g., tumors in which a radiopharmaceutical has been absorbed), using the detector and circuitry of FIGS. 2-3 in coordination with operation of a radiation delivery therapy system, according to aspects of the current invention. According to various aspects, FIG. 4 illustrates an apparatus 400 for detection and imaging of a body portion that has absorbed a radiopharmaceutical, using a directional array 405 that includes a plurality of detectors 100 coordinated with a radiation delivery therapy system 410. According to various aspects, the plurality of detectors 100 may be arranged around a portion of the patient 420 in which a body portion 430 is to be imaged and treated. In an aspect of the disclosure, a triad of detectors 100 may be arranged with their respective collimated apertures, similar to the aperture 120 illustrated above in FIGS. 2A-2B, so as to have the axis of each aperture intersect approximately at a single common coordinate 401 in a defined coordinate space. The patient 420 may be movable on a table 402 in the horizontal plane and vertical direction, for example, while the array 405 may be rotated about a horizontal axis 421 by an angle θ with respect to the single common coordinate 401, to measure radiation intensities in each of the detectors 100 that make up the array 405. By measuring gamma radiation intensities and intensity gradients as the patient is translated horizontally and vertically, and as the array 405 is rotated about the patient, a “brightness” image of the tumor 430 and its location within the patient 420 and relative to the table 402 may be determined. An image may then be constructed by appropriate imaging software in a computing system 450, which is coupled to the array 405 and the table 402, operating to receive data therefrom and to use the data to construct the image. The computing system 450 may also control any spatial movement, such as rotation of the array 405 about the axis 421, while collecting data from the array 405. For example, the rotation of the array 405 about the axis 421 may be accomplished via operation of a motion creating mechanism, such as one or more electric motors operatively engaged with the array 405 and coupled to an electrical output control device. The electrical output control device may, in turn, be coupled to or be part of the computing system 450, which thereby controls the rotation of the array 405.

According to various aspects, in charged particle beam radiation systems, such as proton beam system, a beam 411 may be delivered through a series of focusing and beam bending magnets. A last portion of the beam system may be typically included in a gantry system surrounding the patient 420, in which the beam 411 may be delivered over a semicircle, or even a full circle about the axis 421, where the angular positioning of the gantry, and therefore the direction of projection of the proton beam into the patient 420, may be varied, while always passing substantially through the common coordinate 401, with an additional capability of steering magnets to scan a region around the coordinate 401.

According to various aspects, the array 405 may be similarly mounted on the gantry and aimed so the one or more detectors 100 converge at the same common coordinate 401. The array 405 may be configured about a vertical reference axis that is perpendicular to the horizontal axis 421, and the array 405 may be in a fixed relationship with respect to the portion of the radiation delivery therapy system 410 within the gantry and beam 411. Therefore, both the radiation delivery therapy system 410 and the array 405 may rotate together by the same angle θ about the axis 421 at the common coordinate 401 (e.g., under the control of computer system 450, operating similarly to as described above with regard to rotation of the array 405). Therefore, during contemporaneous operation, both the beam 411 and the collimated apertures of the detectors 100 may have an approximately common intersection point at the tumor.

According to various aspects, the array 405 may further include pairs of detectors 100 (the pairs not being shown in FIG. 4) that may be located facing each other and on opposite sides of the patient to detect pairs of gamma rays from the annihilation of a single positron-electron pair. This dual collection may be beneficial, for example, in improving the signal-to-noise ratio of detected radiation from positron annihilation relative to background radiation (e.g., by removing non-co-incidental background radiation from the received signals). Furthermore, the directional nature of the detectors may further improve both the directional location and position identification of the radiation source. However, it is not required that the detectors 100 be so located on opposite sides of the patient, or that pairs of gamma rays be contemporaneously detected, for example, since it is known that the gamma energy is 511 keV and the detectors 100 have a specific spectroscopic sensitivity to this energy.

According to various aspects, the detectors may be arranged with a known relationship to a radiation beam source, with the patient located relatively statically on the imaging/treatment table. A tumor or other body part, for example, may be imaged by first injecting the patient with an appropriate radiopharmaceutical, such as F-18 FDG. An array of detectors 100, as shown in FIG. 4, may be arranged in a fixed relationship to each other, such as three detectors 100 on a common frame or scaffold, which may be translated and rotated via command from the computer system 450 to detect via triangulation gamma rays emitted by the radiopharmaceutical that is preferentially absorbed by the tumor. In one aspect, the three detectors may have a fixed relationship to each other and thus have a single, approximately or substantially common, point of intersection. When scanned as a unified array by translation of the patient 420 and/or by rotation of the array 405 about the patient, a 3-dimensional map of the tumor may be obtained, based on the signal response of the detectors 100 and image processing performed in the computing system 450, which is coupled to the detectors 100 of the array 405, and the patient's translation table 402. The image intensity and intensity gradients may be correlated with spatial location. In this configuration, all detectors 100 may obtain highly similar intensity readings, since they intersect approximately at one spatial coordinate. At other locations, the detectors 100 may have different intensity responses, indicating that, while one or more detectors 100 may be intersecting the tumor, for example, others are not. Alternative configurations, such as individual directional control of each detector 100 and control of translation of the patient 420 may be used, with appropriate adaptation of a control program running on the computing system 450.

In an aspect of the disclosure, the array of detectors 100 may have a fixed relationship with respect to the radiation beam delivery system, so that the treatment radiation beam intersects approximately or substantially the same intersection point of the collimated acceptance apertures of the detectors. Thus, in one example implementation, the treatment radiation beam 411 may be made operable only when the array of detectors 100 provide signals to the processor that satisfy criteria for tumor identification and location.

According to various aspects, the computing system 450 may be operatively coupled to the therapeutic radiation beam delivery system 410 (e.g., similarly to as discussed above with regard to array rotation). Thus, the therapeutic beam 411, whether gamma rays, proton beam, carbon beam, or any other suitable form of radiation contemplated for tumor treatment, may be directed to a radiation source (such as a body portion that includes a tumor) on the basis of the real-time imaging data acquired by the detectors 100. Furthermore, since the radiation beam delivery system may be rotated while maintaining the same approximate point of intersection at the tumor location, the beam approach direction may be varied to both minimize the damage to normal tissue surrounding the tumor, while passing through and depositing radiation at the body portion location or along a path passing through that location, depending on the form of radiation used.

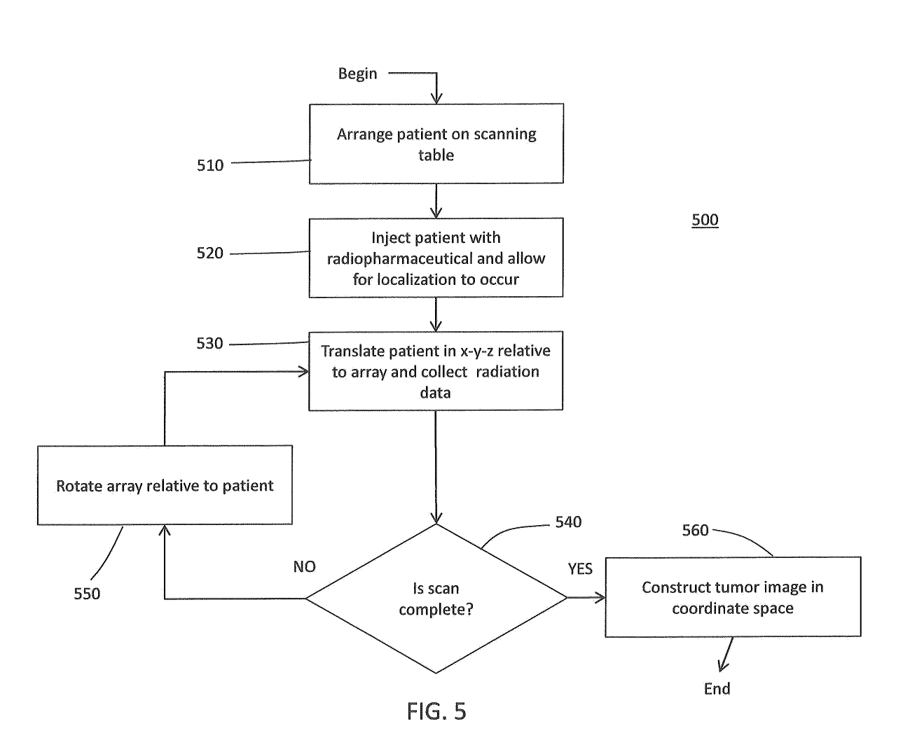

FIG. 5 is a flowchart of an example process of imaging a patient body portion (e.g., tumor) according to aspects of the current invention. The process may begin at 510 by arranging the patient on a scanning table. The patient may then be infused, for example, at 520 with a radiopharmaceutical, such as, for example, FDG, and time may be allowed for the metabolized uptake of the radiopharmaceutical in a body portion of interest, such as a tumor. According to various aspects, the steps 510 and 520 may be interchanged in order. When metabolism of the radiopharmaceutical is sufficient, the patient may be translated on the scanning table, for example, under control of a computing system, and radioactivity level data may be collected from the detectors 100 at 530. According to various aspects, the radioactivity level data may be coordinated with the position of the scanning table, and therefore the location of the tumor with respect to the scanning table may be achieved, for example, via the computing system.

According to various aspects, depending on a prescription selected for scanning (e.g., software determined program of operation), a determination may be made at 540 whether scanning and data acquisition is complete. If the scanning is not complete (a NO decision), then the gantry containing the array may be rotated at 550, so that radioactivity level data may be taken again from a different viewing direction. If the scanning process is complete (a YES decision), the computing system may proceed with constructing an image of the body portion in the common coordinate space of the radiation beam delivery system and array at 560. It may be appreciated that the order of determining whether the scanning is complete, and operation of the rotation of the gantry, may be reversed, i.e., steps 540 and 550 may be interchanged in order.

As a result of the above example process, a three dimensional map of a tumor present in the body of a patient may be constructed to guide delivery of therapeutic radiation to the tumor by combination of controlled positioning of the scanning table and positioning of the radiation delivery therapy system by controlled rotation of the gantry and control of the beam energy and intensity.

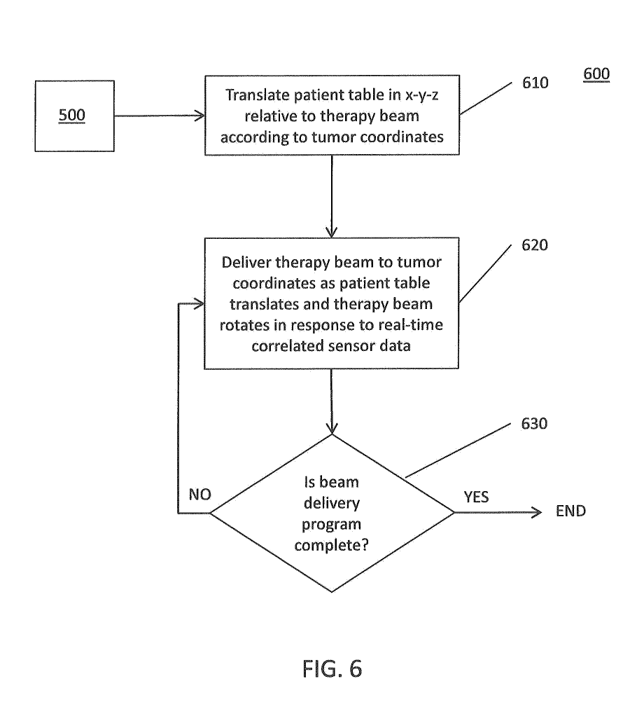

FIG. 6 is an example flowchart for a process of beam therapy delivery guided by imaging detection of an array of one or more CZT collimated detectors, according to aspects of the current invention. According to various aspects, the image coordinate space is provided at 610, where the patient is positioned by translation of the patient table in x-y-z to locate a body portion (e.g., tumor) at the common coordinate through which the therapeutic radiation beam passes. The patient table may be translated at 620 during delivery of the radiation beam to the tumor. The beam delivery system may also have a steering capability, such as steering magnets in a charged particle beam system, so that the beam steering capability may be controlled to sweep the radiation in a pattern corresponding to the body portion shape. At 620, the gantry containing the beam delivery system and the array may also be rotated about the axis that passes through the body location in order to enable the beam to irradiate the body portion (e.g., tumor) from different directions, thus reducing an amount of exposure by tissue not intended to be exposed to such radiation. According to various aspects, the radiation beam may be directed so as to intersect the body portion, and not provide the beam when any portion of the tissue is not located substantially at the common coordinate. Thus, the beam may be pulsed on or off (e.g., by a shutter, beam steering, or other control methods) as needed to minimize damage to tissue not intended to be exposed to the beam. The volume of space irradiated may be monitored at 630 to determine if the body portion is completely scanned. If the delivery of the radiation beam to the body portion is complete—a YES result at 630—no more radiation may be delivered to the region, and the patient's treatment session may be complete. If the program determines that the scanning is not complete—a NO result at 630—control of the beam delivery system and array may be continued by returning to 620 for continued scanning.

According to various aspects, the use of the CZT detector 100 for surgical procedures may involve the following: a radioactive tracer agent emitting a high energy gamma photon administered to a patient prior to surgery. The agent may include but not be limited to F-18 FDG, F-18 FLT, F-18 MISO, F-18 Choline, and C-11 Acetate. The agent may be allowed to localize in a body portion, such as the diseased area (e.g., tumor at location 430 in FIG. 4). A PET scan may be performed to identify the location of the body portion before surgery before the patient being taken to the surgical suite.

In another aspect of the disclosure, one or more detectors may be mounted relative to the patient in a movable fixture permitting the one or more detectors to be both translated in one or more orthogonal axes, and to swivel angularly on controlled gimbals. Thus, a single detector, or a plurality of detectors, may be employed with mobility and with less obtrusive invasion of the operating theater to aid a surgeon in accurately locating the tumor for surgical excision of a cancerous node or tissue.

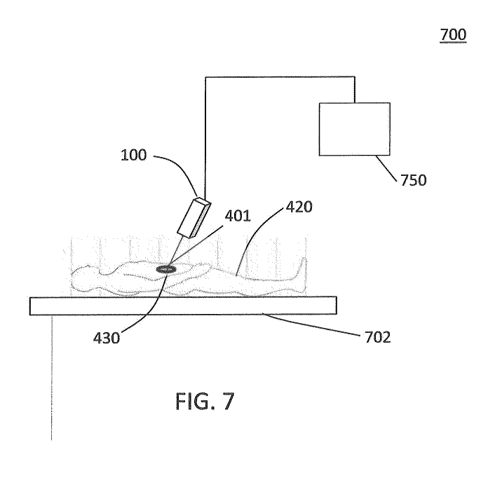

FIG. 7 is a representative diagram of an example apparatus for detecting and imaging tumors with an absorbed radiopharmaceutical using the detector and circuitry of FIGS. 2-3 in coordination with a surgical excision procedure, according to aspects of the current invention. The patient 420 may previously have been scanned diagnostically to identify, locate and image one or more tumors 430 using, for example, PET combined with computed tomography (PET/CT) to image tumors or other body portions 430 (with PET) and denser bone structure (with CT) in order to register body portion locations with respect to bone structure as geographic markers.

According to various aspects, in a surgical theater, for example, separate from the PET/CT facility, there may arise some error for accurate body portion location resulting from the patient being shifted from one facility to another. Where the patient 420 on an operating table 702 has been infused with a radiopharmaceutical, such as F-18 FDG, a detector 100, which may be hand held r mounted in proximity to the patient, may be directed at the patient to directionally detect the tumor 430, which may be a source of radioactive emission. The detector may be coupled to a processing system 750, which may include an indicator, either visual or audible, of the level of radiation measured by the detector 100 to confirm that diseased tissue is being excised from the patient. For example, an audible cue proportional to the detected radiation intensity may be a frequency or an amplitude signal. The cue may be used to guide the surgical procedure to more precisely locate the body portion of the patient (e.g., a tumor). Additionally, the detector 100 may be equipped with a light beam, such as a collimated laser pointer (not shown), to indicate a point on the patient 420 where the detecting “beam” aperture intersects the patient's skin or tissue. The excision instrument and the detector 100 may be integrated into a single hand-held sensing/excision instrument to provide detection feedback to the surgeon as the patient is being operated on.

In another aspect of the disclosure, the detector 100 and scalpel (or other excision instrument) may be manipulated robotically via the processing system 750, similarly to as described above with regard to array rotation. Robotic surgery has advanced to a state of the art where surgical trauma may be minimized and accuracy for more precise tumor removal and preservation of healthy tissue is improved.

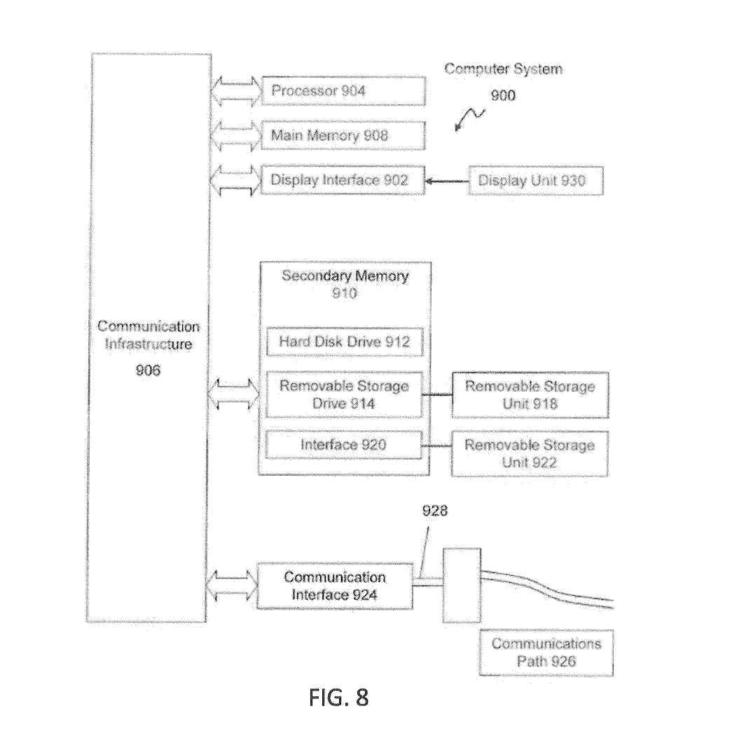

FIG. 8 presents an exemplary system diagram of various hardware components of the computing system 450 and other features, for use in networking the apparatus for detecting and treating tumors, in accordance with an aspect of the present invention. Computer system 900 may include a communications interface 924. Communications interface 924 allows software and data to be transferred between computer system 900 and external devices. Examples of communications interface 924 may include a modem, a network interface (such as an Ethernet card), a communications port, a Personal Computer Memory Card International Association (PCMCIA) slot and card, etc. Software and data transferred via communications interface 924 are in the form of signals 928, which may be electronic, electromagnetic, optical or other signals capable of being received by communications interface 924. These signals 928 are provided to communications interface 924 via a communications path (e.g., channel) 926. This path 926 carries signals 928 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 980, a hard disk installed in hard disk drive 970, and signals 928. These computer program products provide software to the computer system 900. Aspects of the invention are directed to such computer program products.

Computer programs (also referred to as computer control logic) are stored in main memory 908 and/or secondary memory 910. Computer programs may also be received via communications interface 924. Such computer programs, when executed, enable the computer system 900 to perform various features in accordance with aspects of the present invention, as discussed herein. In particular, the computer programs, when executed, enable the processor 910 to perform such features. Accordingly, such computer programs represent controllers of the computer system 900.

In aspects of the invention implemented using software, the software may be stored in a computer program product and loaded into computer system 900 using removable storage drive 914, hard drive 912, or communications interface 920. The control logic (software), when executed by the processor 904, causes the processor 904 to perform various functions in accordance with aspects of the invention as described herein. In another variation, aspects of the invention may be implemented primarily in hardware using, for example, hardware components, such as application specific integrated circuits (ASICs). Implementation of the hardware state machine so as to perform the functions described herein will be apparent to persons skilled in the relevant art(s).

In yet another variation, aspects of the invention may be implemented using a combination of both hardware and software.

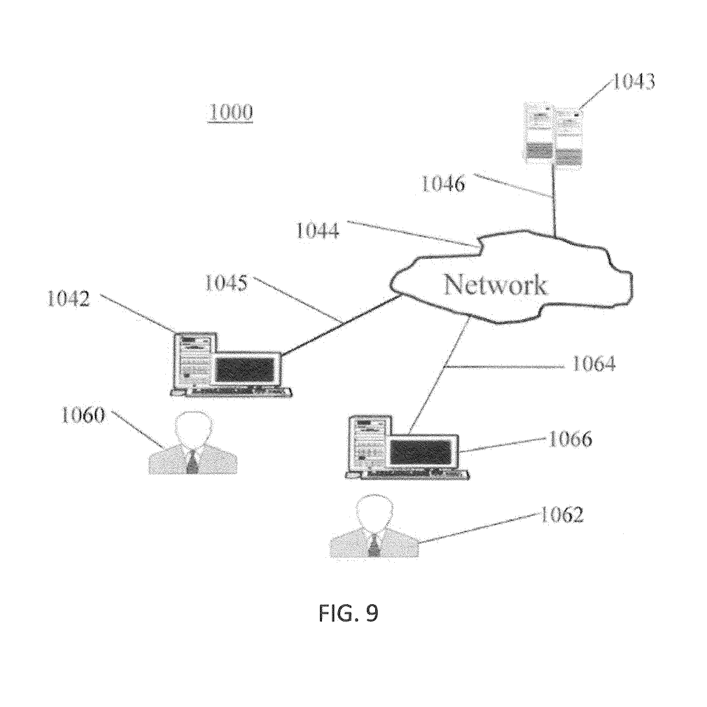

FIG. 9 shows a computer system 1000 on a network, usable with various features in accordance with aspects of the present invention. The computer system 1000 includes one or more accessors 1060, 1062 (also referred to interchangeably herein as one or more “users”) and one or more terminals 1042, 1066. In one variation, data for use in accordance with aspects of the present invention is, for example, input and/or accessed by accessors 1060, 1064 via terminals 1042, 1066, such as personal computers (PCs), minicomputers, mainframe computers, microcomputers, telephonic devices, or wireless devices, such as personal digital assistants (“PDAs”) or a hand-held wireless devices coupled to a server 1043, such as a PC, minicomputer, mainframe computer, microcomputer, or other device having a processor and a repository for data and/or connection to a repository for data, via, for example, a network 1044, such as the Internet or an intranet, and couplings 1045, 1046, 1064. The couplings 1045, 1046, 1064 include, for example, wired, wireless, or fiber optic links. In another variation, the method and system in accordance with aspects of the present invention operate in a stand-alone environment, such as on a single terminal.

The above description is provided to enable any person skilled in the art to fully understand the full scope of the disclosure. Modifications to the various configurations disclosed herein will be readily apparent to those skilled in the art. Thus, the claims are not intended to be limited to the various aspects of the disclosure described herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. A claim that recites at least one of a combination of elements (e.g., “at least one of A, B, or C”) refers to one or more of the recited elements (e.g., A, or B, or C, or any combination thereof). All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A gamma ray detector, comprising:
an elongated rod configured to react with gamma rays to emit a signal;
a casing encapsulating the elongated rod, the casing including an aperture at an end of the casing with respect to a longitudinal axis of the elongated rod; and
a pair of electrodes configured to apply a charge to the elongated rod.

2. The gamma ray detector of claim 1, wherein the elongated rod comprises cadmium zinc telluride (CZT).

3. The gamma ray detector of claim 1, wherein the casing is configured to shield the elongated rod from gamma rays emitted in directions at least partially transverse to the longitudinal direction.

4. The gamma ray detector of claim 1, wherein the aperture is configured to allow primarily gamma rays that are incident along the longitudinal direction of the elongated rod to react with the elongated rod.

5. The gamma ray detector of claim 1, wherein gamma ray detection is enhanced along the longitudinal axis by substantially collimating in a longitudinal direction via the aperture and the casing.

6. A gamma ray detector system comprising:
one or more gamma ray detectors, each of the one or more gamma ray detectors including;
an elongated rod configured to react with gamma rays to emit a signal;
a casing encapsulating the elongated rod, the casing including an aperture at an end of the casing with respect to a longitudinal axis of the elongated rod; and
a pair of electrodes configured to apply a charge to the elongated rod;
wherein the one or more gamma ray detectors are arranged in an array about a target volume;
a scanning device coupled to the one or more detectors to control a coordinated scanning activity of the sensors relative to the target volume; and
a processing device coupled to the one or more gamma ray detectors and the scanning device, wherein the processing device is configured to assemble and output at least one of a three-dimensional image and coordinate location of the concentration of radionuclide.

7. The gamma ray detector system of claim 6, wherein:
the target volume includes a concentration of a radiation emitting radionuclide, and the longitudinal direction of the one or more detectors intersect at a common spatial coordinate within the target volume.

8. The system of claim 6, wherein the array comprises two detectors located facing each other.

9. The system of claim 6, further comprising:
a charge amplifier coupled to each detector;
an analog to digital converter (ADC) coupled to the charge amplifier;
a pulse height counter coupled to the ADC; and
a pulse counter coupled to the ADO;
wherein the processing device acquires a three-dimensional image on the basis of a signal received from the pulse counter and the volume of space scanned by the one or more sensors.

10. The system of claim 9, wherein the processing device comprises a pulse processor circuit that discriminates 511 keV gamma emissions.

11. A tissue treatment apparatus comprising:
one or more collimated detectors, each of the one or more collimated detectors including:
an elongated rod configured to react with gamma rays to emit a signal;
a casing encapsulating the elongated rod, the casing including an aperture at an end of the casing with respect to a longitudinal axis of the elongated rod; and
a pair of electrodes configured to apply a charge to the elongated rod;
wherein each of the one or more collimated detectors is configured to intersect signals received from a known coordinate and measure gamma radiation activity from the tissue located thereat; and
a radiation delivery device configured to direct radiation through the known coordinate on the basis of the measured gamma radiation activity to the tissue at the known coordinate.

12. The apparatus of claim 9, wherein the one or more collimated detectors and the radiation delivery devices are in a static spatial relationship relative to one another.

13. A method of mapping a body portion of a patient, the method comprising:
positioning the patient in a first fixed spatial location;
injecting the patient with a radiopharmaceutical;
detecting radioactivity level emitted from one or more portions of the patient;
determining a radioactivity level that corresponds to the body portion of the patient to be mapped;
moving the patient in one or more other fixed spatial locations;
detecting radioactivity level corresponding to each of the one or more other fixed spatial locations; and
mapping the body portion of the patient on the basis of the detected radioactivity levels corresponding to the first fixed spatial location and corresponding to the one or more fixed spatial locations.

14. The method of claim 13, wherein the first fixed spatial location is a horizontal position.

15. The method of claim 13, wherein the one or more fixed spatial locations correspond to at least one a horizontal movement, a vertical movement, a lateral movement, and an angular movement of the patient.

16. A method of treating tissue, comprising:
mapping the tissue according to claim 13, the tissue including a tumor; and
delivering a therapeutic beam to the tissue located via the mapping.

17. The method of claim 16, wherein delivering the therapeutic beam to the tissue takes place in an intra-operative surgical environment.

18. A system for mapping a tumor in a patient, comprising:
a processor;
a user interface functioning via the processor; and
a repository accessible by the processor; wherein the patient is positioned in a first fixed spatial location;
the patient is injected with a radiopharmaceutical;
a radioactivity level emitted from one or more portions of the patient is detected;
a radioactivity level that corresponds to a tumor is determined;
the patient is moved in one or more fixed spatial locations;
another radioactivity level corresponding to each of the one or more fixed spatial locations is determined; and
the tumor is mapped on the basis of the detected radioactivity levels corresponding to the first fixed spatial location and corresponding to the one or more fixed spatial locations.

19. A system for mapping a tumor in a patient, comprising:
means for positioning the patient in a first fixed spatial location;
means for injecting the patient with a radiopharmaceutical;
means for detecting radioactivity level emitted from one or more portions of the patient;
means for determining a radioactivity level that corresponds to a tumor;
means for moving the patient in one or more other fixed spatial locations;
means for detecting radioactivity level corresponding to each of the one or more other fixed spatial locations; and
means for mapping the tumor on the basis of the detected radioactivity levels corresponding to the first fixed spatial location and corresponding to the one or more fixed spatial locations.

20. A computer program product comprising a computer usable medium having control logic stored therein for causing a computer to map a body portion of a patient, the control logic comprising:
computer readable program code means for positioning the patient in a first fixed spatial location;
computer readable program code means for injecting the patient with a radiopharmaceutical;
computer readable program code means for detecting radioactivity level emitted from one or more portions of the patient;
computer readable program code means for determining a radioactivity level that corresponds to the body portion of the patient to be mapped;
computer readable program code means for moving the patient in one or more other fixed spatial locations;
computer readable program code means for detecting radioactivity level corresponding to each of the one or more other fixed spatial locations; and
computer readable program code means for mapping the tumor on the basis of the detected radioactivity levels corresponding to the first fixed spatial location and corresponding to the one or more fixed spatial locations.
